(19) United States
(12) Patent Application Publication
(10) Pub. No.: US 2020/0230402 A1
(43) Pub. Date: Jul. 23, 2020

(54) LEAD ELECTRODE WITH IMPROVED MRI CONDITIONALITY

(71) Applicant: BIOTRONIK SE & CO. KG, Berlin (DE)

(72) Inventors: JEFFREY A. VON ARX, LAKE OSWEGO, OR (US); ROBERT R. SHURIG, TUALATIN, OR (US); KRISHNA K. N. KURPAD, PORTLAND, OR (US)

(21) Appl. No.: 16/711,495

(22) Filed: Dec. 12, 2019

Related U.S. Application Data
(60) Provisional application No. 62/793,916, filed on Jan. 18, 2019.

(51) Int. Cl. A61N 1/08; A61N 1/05

(52) U.S. Cl. CPC A61N 1/086; A61N 1/375; A61N 1/0551

(57) ABSTRACT

A lead electrode has a lead body extending along a longitudinal axis, and a tubular shield arranged around at least a part of the lead body. The shield is configured to protect the lead electrode from electromagnetic interference. The shield is formed with a plurality of slots arranged along the longitudinal axis of the lead electrode or the shield is formed with at least one helical slot which extends along a helical path along the longitudinal axis.

### CROSS-REFERENCE TO RELATED APPLICATION

This application claims the benefit, under 35 U.S.C. § 119(e), of provisional application No. 62/793,916, filed Jan. 18, 2019; the prior application is herewith incorporated by reference in its entirety.

### BACKGROUND OF THE INVENTION

### Field of the Invention

The present invention relates to a lead electrode, particularly for therapeutic electrostimulation, as for instance spinal cord stimulation (SCS).

Magnetic resonance imaging (MRI) machines put out very large radio frequency (RF) fields during operation. The energy in these fields is picked up by conductors in implantable medical device leads, such as lead electrodes used for neurostimulation, such as spinal cord stimulation (SCS), vagus nerve stimulation (VNS), deep brain stimulation (DBS).

If such leads are not adequately shielded from these fields or do not comprise mechanisms for dissipating this energy, then the energy may go to two places, both of which can cause problems. First, a good portion of the picked-up energy goes to the electrodes of the lead where it is dissipated as heat. Disadvantageously, such electrode heating can cause damage to the tissue of the patient. Second, the remaining energy may go back to the implantable pulse generator, where it can damage the sensitive circuitry.

Conventional neuro leads (straight wire conductors) are suitable for MRI conditionally safe labeling in normal operating mode (SAR of W/Kg) only with an exclusion zone, or at very low MR RF powers. Furthermore, leads comprising labeling allowing full body scanning at 2 W/kg usually comprise a complex and bulky structure.

Furthermore, published U.S. patent application US 2012/035697 A1 discloses wrapping a foil around the lead so as to have helical slots in the foil for the reduction of mechanical stiffness of the entire lead.

Further, published U.S. patent application US 2015/314123 discloses a medical lead having a plurality of inner conductors, wherein each inner conductor may have an outer layer that can be formed by a laser-cut tube.

### SUMMARY OF THE INVENTION

In the present description of the invention, the term “lead electrode” is used synonymously for a medical lead, having, inter alia, a lead body and one or more electrodes for delivering electrical stimulation, wherein here the term “electrode” is used as a synonym.

It is accordingly an object of the invention to provide a lead electrode, which overcomes the above-mentioned and other disadvantages of the heretofore-known devices and methods of this general type and which provides for a lead electrode that is MR conditional and comprises a relatively simple design.

With the above and other objects in view there is provided, in accordance with the invention, a lead electrode, comprising:

a lead body extending along a longitudinal axis of the lead electrode;

a tubular shield arranged around at least a portion of the lead body, the shield being configured to protect the lead body from electromagnetic interference;

the shield being formed with a plurality of slots arranged along the longitudinal axis of the lead electrode; or

the shield being formed with at least one helical slot which extends along a helical path along the longitudinal axis.

In other words, the objects of the invention are achieved, in accordance with the invention, by a lead electrode that comprises a lead body extending along a longitudinal axis, and a tubular shield arranged around at least a part of the lead body. The shield is configured to protect the lead electrode from electromagnetic interference (EMI) and the shield is formed with a plurality of slots arranged side by side along the longitudinal axis of the lead electrode or the shield comprises at least one helical slot which extends along a helical path along the longitudinal axis.

Particularly, according to an embodiment of the lead electrode, the respective slot extends in the peripheral direction of the shield.

According to an embodiment of the inventive lead electrode, the plurality of slots are arranged side by side along the longitudinal axis of the lead electrode.

Furthermore, according to an embodiment of the lead electrode, the respective slot forms an arc.

Moreover, according to an embodiment of the present invention, the slots comprise at least one slot having the shape of a circle segment, or wherein the respective slots comprise a helical slot, or wherein the respective slots comprise interrupted helical slots (35).

According to an alternative embodiment, the respective slot is a helical slot. The respective helical slot can comprise a pitch. The pitch of the different slots can vary, particularly so as to influence the mechanical properties of the shield along the longitudinal axis.

According to an embodiment of the present invention, the respective slot is an interrupted helical slot in which the slot takes a helical path. According to this embodiment, the slot begins and ends at intervals such that the slot is not one continuous helical cut. The respective helical path can comprise a pitch. The pitch of the different slots can vary, particularly so as to influence the mechanical properties of the shield along the longitudinal axis.

In an embodiment of the present invention, the slot is one helical slot, which spirals around at least a part of the length of the shield. According to an embodiment, the helical slot spirals around the entire length of the shield.

In an embodiment of the present invention, the tubular shield comprises at least one slot having a zigzag shape or an S-shape. In an embodiment, the tubular shield comprises at multiple slots and at least one cutout, wherein the cutout is arranged between two slots. The cutout can have a round or rectangular shape. According to an embodiment, the tubular shield comprises multiple cutouts, wherein the cutouts are arranged in a regular pattern. According to an embodiment, the tubular shield comprises cutouts forming a mesh. For instance, the cutouts are manufactured using laser-cutting procedures.

Furthermore, according to an embodiment of the lead electrode, the slots are grouped in pairs of slots (particularly in case each slot is circular arc shaped), wherein said pairs are arranged side by side along the longitudinal axis, wherein each pair of slots comprises a first and a second slot, wherein the first and the second slot are arranged side by side in the peripheral direction.

Particularly, the first slot and second slot of each pair of slots preferably each form a circular arc comprising the same arc length, wherein the respective arc length is smaller or greater than 180°. An arc having an arc length greater than 180° has the advantage that it allows for overlap between each pair of slots which are located next to each other having an offset along the longitudinal axis. Such overlap would increase the flexibility of the tubular shield.

Furthermore, according to a preferred embodiment, the two slots of each pair of slots are arranged offset in the peripheral direction with respect to the two slots of a neighboring pair of slots.

Furthermore, according to an embodiment of the lead electrode, the slots are laser-cut into the shield, or wherein the slots are etched into the shield.

Furthermore, according to an embodiment of the lead electrode, the shield comprises or is formed out of one of the following materials: a metal, a metallic alloy, stainless steel; an alloy comprising Ni, Co, Cr, and Mo (e.g. MP35N); cobalt-chromium (CoCr); gold; tantalum.

Particularly, the shield is a metallic shield. Such metallic tubes with laser-cut slots are also known as hypotubes. Particularly, according to an embodiment of the present invention, the tubular shield is a hypotube.

Furthermore, according to an embodiment of the lead electrode, the shield comprises an outer surface, wherein the outer surface is coated with gold, particularly electroplated with gold, and/or wherein the shield comprises an inner surface facing the lead body, wherein the inner surface is coated with gold, particularly electroplated with gold.

Furthermore, according to an embodiment of the lead electrode, the lead electrode comprises an electrically insulating layer arranged on the shield. Particularly, the electrically insulating layer is configured to block DC current conduction and to allow high frequency energy transfer.

Furthermore, according to an embodiment of the lead electrode, the electrically insulating layer comprises at least one through-opening or several through-openings to establish electrical contact between the shield and surrounding tissue and to allow dissipation of RF energy induced during magnetic resonance imaging (MRI) of the lead electrode to the surrounding tissue through the uninsulated sections of the shield.

Particularly, according to an embodiment, the tubular shield forms a mechanical structure configured to add hoop strength and to reduce local stress in the lead body at points of bending. Particularly, according to an embodiment, the shield comprises a varying flexibility along its length (i.e. along the longitudinal axis) to allow optimization of flexibility/strain relief/production cost.

Furthermore, according to an embodiment of the lead electrode, the electrically insulating layer comprises one or more of the following materials or is formed out of one or more of the following materials: polyurethane, silicone, silicon carbide, parylene silicone-urethane copolymer. According to an embodiment of the lead electrode, the insulating layer can comprise a thickness in the range to 150 μm. Some insulation layers (e.g. silicon carbide, parylene) have the additional benefit of reducing metal ion migration from the hypotube shield, and thereby reduce the likelihood of chronic degradation to other electrically insulating layers.

Furthermore, according to an embodiment of the lead electrode, the tubular shield comprises at least one marker for identifying the lead electrode (e.g. the marker codes information about the lead electrode), wherein the marker is adapted such that it is visible under fluoroscopy. This can be used to easily identify the lead electrode under fluoroscopy as an MR conditional model. Particularly, the marker forms a through-opening formed in the tubular shield.

Furthermore, according to an embodiment of the lead electrode, the lead body is formed out of or comprises an electrically insulating material. Particularly, said material is one of or comprises one of: polyurethane, silicone, or silicone-urethane copolymer. Particularly the polyurethane, silicone, or silicone-urethane copolymer can be extruded to form the lead body (including the lumens described below). Alternatively, according to an embodiment of the present invention, the polyurethane, silicone, or silicone-urethane copolymer may be applied to the shield via a dip-coating process.

Furthermore, according to an embodiment of the lead electrode, the lead body comprises a center lumen configured to receive a longitudinal element (e.g. a stylet or guide wire). The center lumen can have an inner diameter of e.g. 0.45 mm.

Furthermore, according to an embodiment of the lead electrode, the lead body comprises (besides the center lumen) a plurality of lumens wherein the respective lumen extends along the longitudinal axis. Particularly, these lumens are arranged around the center lumen. Further, said plurality of lumens consists of eight lumens. Particularly, the respective lumen can have an inner diameter of e.g. 0.25 mm.

Furthermore, according to an embodiment of the lead electrode, the lead electrode comprises a plurality of conductors. Particularly, the conductors are electrically connected to electrodes and the connector contacts of the lead electrode. Particularly, the respective conductor can have an outer diameter in the range from 0.13 mm to 0.2 mm. The complete lead electrode itself can have an outer diameter of e.g. 1.33 mm (4 Fr).

Furthermore, according to a preferred embodiment, the lead electrode comprises eight conductors.

Furthermore, according to an embodiment of the lead electrode, each conductor is coated with an electrical insulation. The insulation can be formed or can comprise one of the following materials: Ethylene tetrafluoroethylene (ETFE, CAS Number 25038-71-5), perfluoroalkoxy alkane (PFA), polytetrafluoroethylene (PTFE).

Furthermore, according to an embodiment of the lead electrode, each conductor is arranged in one of the lumens.

Furthermore, according to an embodiment of the lead electrode, the lead electrode or the shield comprises a length in the direction of the longitudinal axis that lies in the range from 35 cm to 95 cm, or more.

Other features which are considered as characteristic for the invention are set forth in the appended claims.

Although the invention is illustrated and described herein as embodied in a lead electrode with improved MRI conditionality, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 shows an embodiment of a shield of a lead electrode according to the present invention, wherein the shield contains paired circular arc slots;

FIG. 2 shows a cross sectional view of an embodiment of a lead electrode according to the present invention, wherein the cross section shown extends perpendicular to the longitudinal axis (X) shown in FIG. 1;

FIG. 3 shows SAR measurements at different points along a distal tip of a lead electrode according to the present invention, wherein the eight peaks correspond to the eight electrodes of the lead electrode; and

FIG. 4 shows SAR measurements for the lead electrode of FIG. 3, but without the shield;

FIG. 5 shows an embodiment of a shield of a lead according to the present invention, wherein the shield contains interrupted helical slots;

FIG. 6A shows the distal end of a lead electrode with 8 electrodes and shield according to an embodiment of the invention; and

FIG. 6B is a close-up of the distal end of the lead electrode according to FIG. 6A.

### DETAILED DESCRIPTION OF THE INVENTION

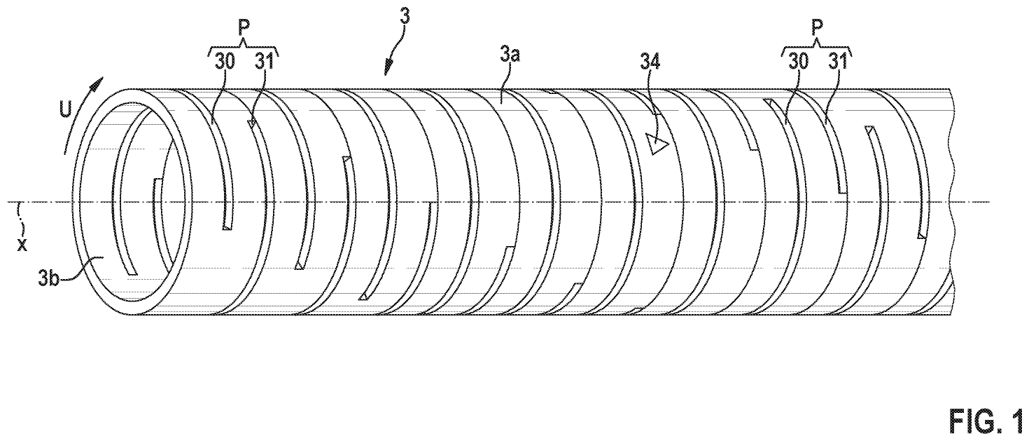

Referring now to the figures of the drawing in detail and first, particularly, to FIG. 1 in conjunction thereof, there is shown an embodiment of a lead electrode 1 according to the present invention. Particularly, the lead electrode 1 is configured to apply neurostimulation to a patient, particularly spinal cord stimulation (SCS).

The lead electrode 1 comprises a lead body 2 extending along a longitudinal axis x, and a tubular shield 3 arranged around the lead body 2 in a peripherally circumferential direction U of the shield 3, wherein the shield 3 is configured to protect the lead conductors 4 from electromagnetic interference (EMI), and wherein the shield 3 comprises a plurality of slots 30, 31 arranged side by side along the longitudinal axis x of the lead electrode 1. Preferably, the slots 30, 31 are laser-cut into the metallic shield 3.

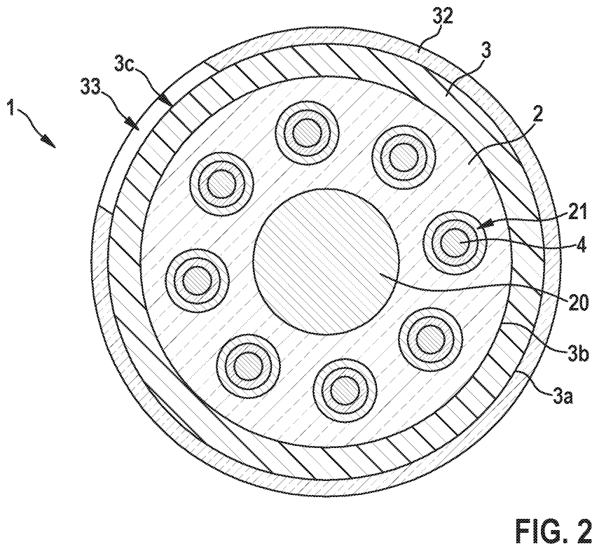

In a preferred embodiment the lead electrode 1 (as shown in FIG. 2) has eight conductors 4 connected to eight electrodes (not shown), and all eight conductors 4 are enclosed by the shield 3, which serves to shield the conductors 4 from EMI, particularly EMI from MRI machines.

Particularly, according to an embodiment, the tubular shield 3 is formed by a so-called hypotube, which is usually a metallic tube (cf. FIG. 1) comprising laser-cut slots 30, 31 to make it flexible. Such a tube is ideal for shielding a lead electrode 1 because it can be made from tubing with thin walls (adding minimal bulk to the lead 1), and can be made extremely flexible. In a preferred embodiment the shield (e.g. hypotube) 3 comprises a wall thickness in the range from 50 μm to 100 μm.

A preferred cut pattern of the slots 30, 31 is shown in FIG. 1. According thereto, the respective slot 30, 31 extends in the peripheral direction U of the shield 3 (i.e. around the longitudinal axis x). Particularly, the slots 30, 31 are grouped in pairs P of slots 30, 31, wherein said pairs P are arranged side by side along the longitudinal axis x, wherein each pair P of slots comprises a first and a second slot 30, 31, wherein the first and the second slot 30, 31 are arranged side by side in the peripheral direction U. Preferably, the first slot 30 and second slot 31 of each pair P of slots preferably each form a circular arc comprising preferably the same arc length, wherein the respective arc length is greater than 180°.

According to an embodiment of the present invention, the tubular shield comprises at least two slots, wherein each slot extends in the peripheral direction U of the shield 3, each slot forming an arc of less than 180 degrees. Preferably, each slot extends in a plane perpendicular to the x-axis, wherein the crossing point of said plane and the x-axis is the same for the at least two slots. According to an embodiment, the shield comprises two slots which are located at the same position on axis x.

According to a preferred embodiment, the two slots (i.e. the first and the second slot 30, 31) of each pair P of slots are arranged offset in the peripheral direction with respect to the two slots 30, 31 of a neighboring pair P of slots, as can be seen from FIG. 1.

As an alternative to circular arc shaped slots 30, 31, the slots can be helical slots comprising a pitch that can vary along the lead electrode 1 (see also below).

Particularly, cut shields 3 can be made by laser cutting a tube or by etching. Laser cutting is the preferred manufacturing method. Hypotubes/shields 3 can, in principle, be made of any biocompatible material, but for use as MRI shielding, high conductivity material is preferred according to an embodiment. According to a preferred embodiment of the invention the shield (e.g. hypotube) is made of stainless steel. In other embodiments MP35N, Cobalt Chromium, Nitinol, or tantalum can also be used. Furthermore, electroplating of the shield 3 with a good conductor such as gold can be used to improve conductivity and hence shielding performance, while retaining biocompatibility. The conductivity of stainless steel is ˜1.3×10⁶ S/m, whereas the conductivity of gold is ˜44.2×10⁶ S/m, or ˜33 times higher than stainless steel. Therefore, a gold coating can significantly improve the conductivity and hence the performance of the shield 3. According to an embodiment, the shield 3 (e.g. laser cut hypotube) is 75 μm in wall thickness, and coated on its outer surface 3a and its inner surface 3b with an e.g. 10 μm thick layer of electroplated gold.

Particularly, FIG. 2 shows a cross section of an embodiment of the proposed lead electrode 1. Here, the shield or hypotube 3 can be insulated with an electrically insulating layer 32 up to 150 μm thick formed e.g. out of polyurethane.

Such a polyurethane coating 32 can either be obtained by dipping the shield or hypotube 3, or it can be a pre-extruded polyurethane tube 32. In an alternative embodiment the electrically insulating layer 32 is silicone. In yet another embodiment the electrically insulating layer 32 is silicon carbide (e.g. Biotronik's ProBIO coating). In yet another embodiment the electrically insulating layer 32 is parylene. In yet another embodiment the electrically insulating layer 32 is silicone-urethane copolymer. Inside the shield or hypotube 3 is the lead body 2 that forms an inner insulation (e.g. out of polyurethane), which can be formed (particularly extruded) with e.g. nine lumens, a center lumen 20 and eight lumens grouped around the center lumen (in an alternative embodiment the lead electrode may comprise a single central lumen 20, and a single ring shaped cavity surrounding the center lumen that contains the individually insulated conductors 4). The center lumen 20 can have an inner diameter of e.g. ˜0.45 mm (this center lumen 20 is configured for insertion of a stylet, e.g. during implantation of the lead electrode 1). The eight outer lumens 21 are for each of the eight conductors (e.g. cables) of the lead electrode, and can comprise an inner diameter of e.g. ˜0.25 mm. Each of the eight conductors 4 can comprise an outer diameter of e.g. 0.075 mm, 0.1 mm, 0.13 mm to 0.20 mm. In an embodiment, each conductor 4 is coated with insulation e.g. ETFE or PFA or PTFE. Regarding the specific dimensions stated above, the outer diameter of the finished lead electrode 1 can amount to 1.33 mm (4 Fr).

Furthermore, in a preferred embodiment, the shield 3 (e.g. hypotube) is configured to be electrically connected to tissue of the patient at either the proximal end (just distal to a header connection), the distal end (just proximal to the electrodes of the lead 1), or at both ends. Electrical connection to tissue allows the shield 3 to better dissipate the MRI induced RF energy that it picks up into tissue (although at the MRI RF frequency significant dissipation can be achieved by capacitive coupling through the insulation). In a preferred embodiment the electrical connection to tissue is made by a section of the shield 3 (e.g. laser cut hypotube) itself being exposed to the body (e.g. there is no outer insulation 32 over that section). This exposed section should be significantly larger in area than the lead's 1 electrodes (i.e. >10× the surface area) so that the RF induced heating of the shield/tissue interface is minimized.

In a further embodiment, the electrical connection to tissue is achieved by a large flexible electrode comprised by the lead electrode 1 (similar to an ICD shock coil). This embodiment makes for a more complicated lead construction (due to weld joints being needed between the shield 3 (e.g. hypotube) and the energy dissipating electrode), but it does allow for a superior electrode material to be used for the RF energy dissipating electrode (such as platinum or platinum-iridium) while allowing the shield 3 (e.g. hypotube) to be MP35N, stainless steel, cobalt chromium, nitinol, or tantalum.

In a further embodiment, proximal and distal sections of the shield 3 (e.g. hypotube) that particularly meet the 10× surface area criterion above, are located a small distance away from the ends of the shield 3 (e.g. hypotube) and are exposed to the surrounding tissue while the ends of the shield 3 (e.g. hypotube) are insulated from the surrounding tissue. This embodiment ensures that the ends of the shield 3 (e.g. hypotube) where E-fields are typically concentrated, are not in direct contact with tissue.

In a further embodiment, one or more additional sections of the shield 3 (e.g. hypotube) that particularly also meet the 10× surface area criterion above, are located between said exposed proximal and distal sections of the above-described embodiment. Exposed sections of the shield 3 (e.g. hypotube) typically represent loss regions, which may also be interpreted as lumped high resistance regions of the shield 3 (e.g. hypotube) that effectively suppress the formation of standing waves on the shield 3.

In a further embodiment/arrangement comprising a lead electrode 1 according to the present invention, the shield 3 (e.g. hypotube) is electrically connected to a casing of an implantable pulse generator (IPG). The casing can be made out of titanium. Here, the casing acts as at least one of the RF energy dissipating electrodes for the shield 3. The advantage of this embodiment is that it takes advantage of the large surface area of the IPG casing to dissipate the MRI induced energy from the shield 3. In a preferred implementation of this embodiment, the proximal end of the shield 3 is mechanically and electrically connected to an anchor ring that is located just distal to proximal terminal connectors of the lead electrode 1. The lead 1 can be commonly secured to the IPG at the anchor ring by a set screw assembly located in an IPG header. Particularly, the metallic (conductive) set screw housing in the IPG header is electrically connected to the casing, thus establishing electrical contact between the shield 3 and the casing.

In a further embodiment, the shield 3 (e.g. hypotube) is floating from a DC perspective. In this embodiment the shield 3 is not electrically connected at low frequencies at either the proximal or distal end. However, at high frequencies (e.g. the 64 MHz and 128 MHz RF frequencies of MR machines) the electrically insulating layer 32 results in a high degree of capacitive coupling (and therefore energy transfer) to the tissue. The layer 32 provides some advantages over a completely bare, electrically exposed shield 3. First, the layer 32 provides a lubricious surface to ease implantation. Second, the electrically insulating layer 32 protects against tissue ingrowth into the shield 3. Third, the layer 32 blocks potentially harmful DC current conduction during medical procedures such as defibrillation.

Particularly, the shield 3 (e.g. hypotube) covers the lead body 2 for the majority of its length in the direction of the longitudinal axis x. Preferably, it does not cover the proximal connector contacts, and it does not cover the electrodes at the distal end of the lead for obvious reasons (so that the electrodes can electrically contact tissue for applying stimulation), but in a preferred embodiment the shield 3 comes within approximately 1 cm of these structures. In a preferred embodiment the lead 1 is iso-diametric, with thicker sections of the layer 32 (polyurethane insulation) at the ends where there is no shield 3 (thus making up the excess volume).

In addition to shielding the lead conductors 4, the shield 3 (e.g. hypotube) adds hoop strength and strain relief to the lead body 2. This protects the conductors 4 at anchoring sites and other pinch points. A pitch of the slots 30, 31 can vary along the length of the shield to yield a desired flexibility profile. The pitch is understood as the distance between two neighboring slots, wherein the distance is measured in axial direction of the lead electrode. In general, the more flexible a section of the shield 3, the less strain relief in that area, so varying the cut pitch can optimize the flexibility/strain relief along the lead's length (i.e. along the longitudinal axis x). Also, the higher the cut pitch, the more cutting of the shield 3 is required, which generally results in a more expensive finished shield 3. Therefore, varying the (cut) pitch of the slots can also optimize manufacturing costs. Also, creating more flexible sections at each end of the shield 3 can reduce local stresses in the lead body 2 at these shield ends.

Furthermore, the shield 3 (e.g. hypotube) can also serve as a fluoroscopy identifying marker for the lead 1. An identifier can be cut right into the shield 3. This could be used to easily identify the lead under fluoroscopy as an MR conditional model.

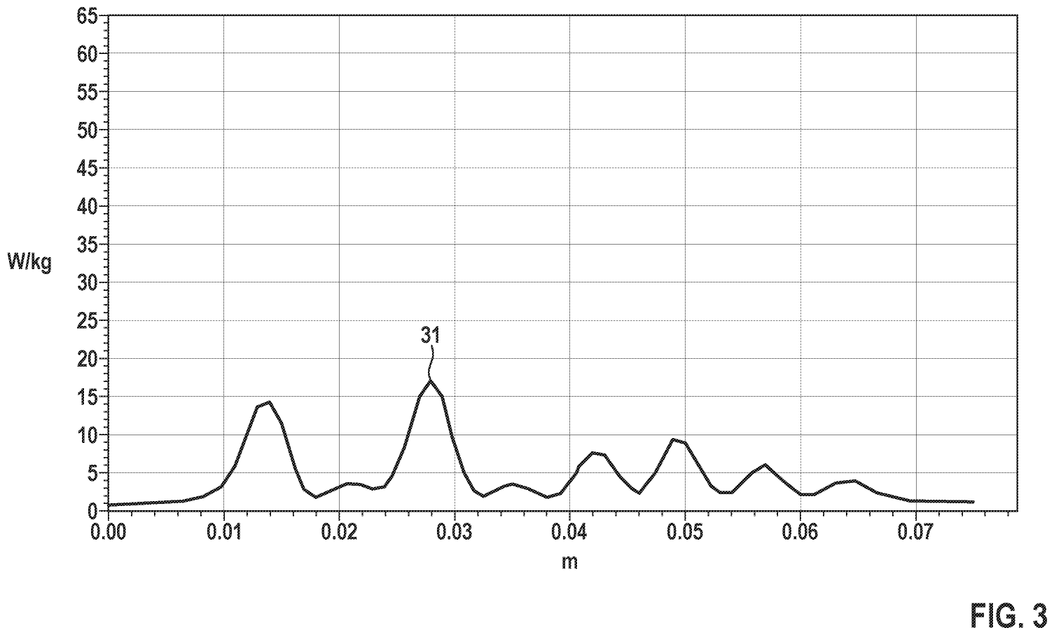

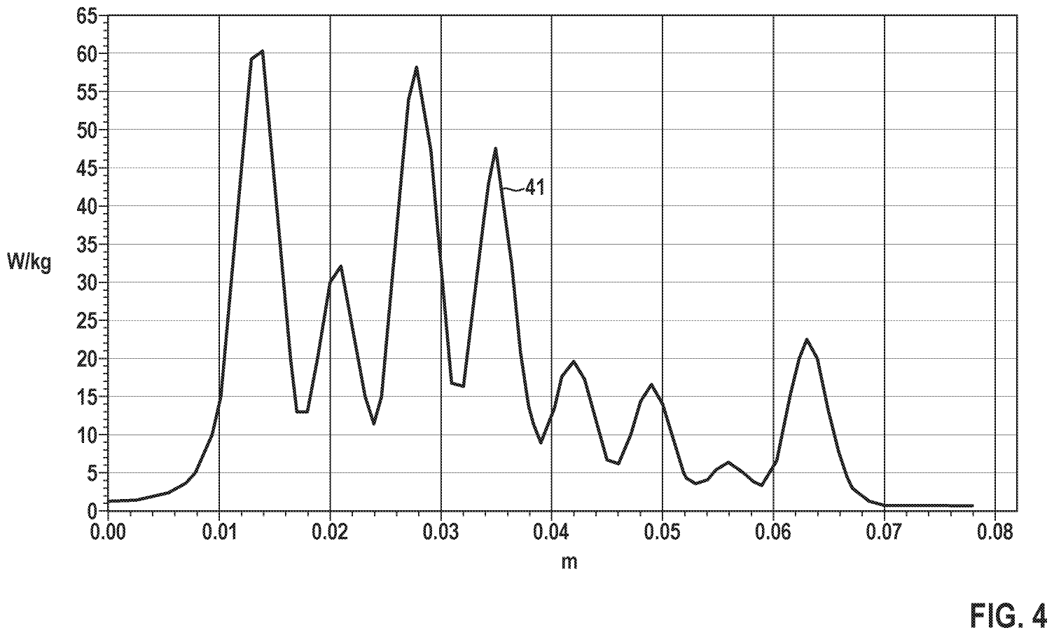

Graph 31 represents test results with prototype leads 1 made with the proposed shield 3 (e.g. hypotube) are shown in FIG. 3, which shows SAR measurements along the distal portion of the lead comprising a shield 3 with slots. In the graph, the y-axis corresponds to the measured SAR value in W/kg, wherein the x-axis corresponds to the lead position along the lead body. In contrast thereto, FIG. 4 shows graph 41 which depicts test results for SAR measurements for the same lead 1 without the shield 3. In these Figures each of the eight peaks corresponds to the position of an electrode. As can be seen from FIGS. 3 and 4, the shield 3 reduces SAR values, and therefore heating by a factor of 3.

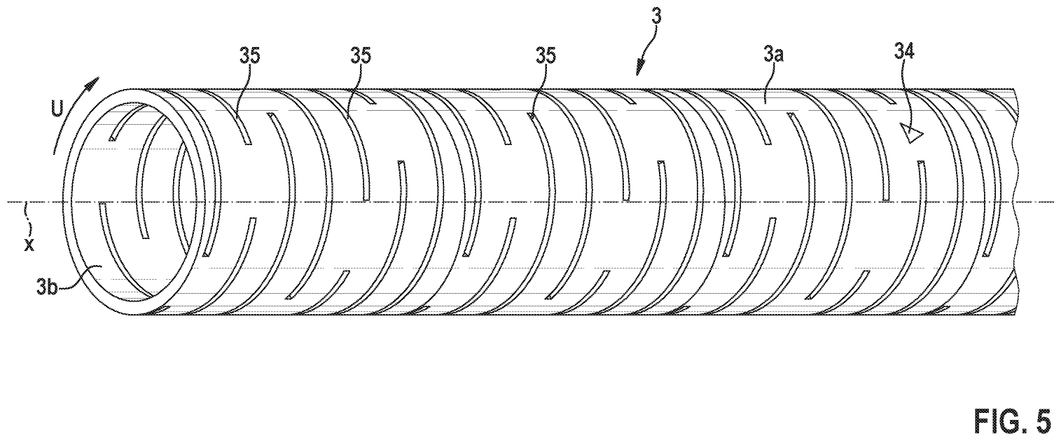

FIG. 5 shows an embodiment of the present invention of a shield of a lead electrode, wherein the shield contains interrupted helical slots. As an alternative to circular arc shaped slots 30, 31, or a continuous helical slot, the slots can be interrupted helical slots 35. In this embodiment, the slots take a helical path. According to an embodiment, the slots begin and end at intervals such that the shield comprises more than one interrupted helical cut. The pitch of the different helical slots can vary, particularly so as to influence the mechanical properties of the shield along the longitudinal axis.

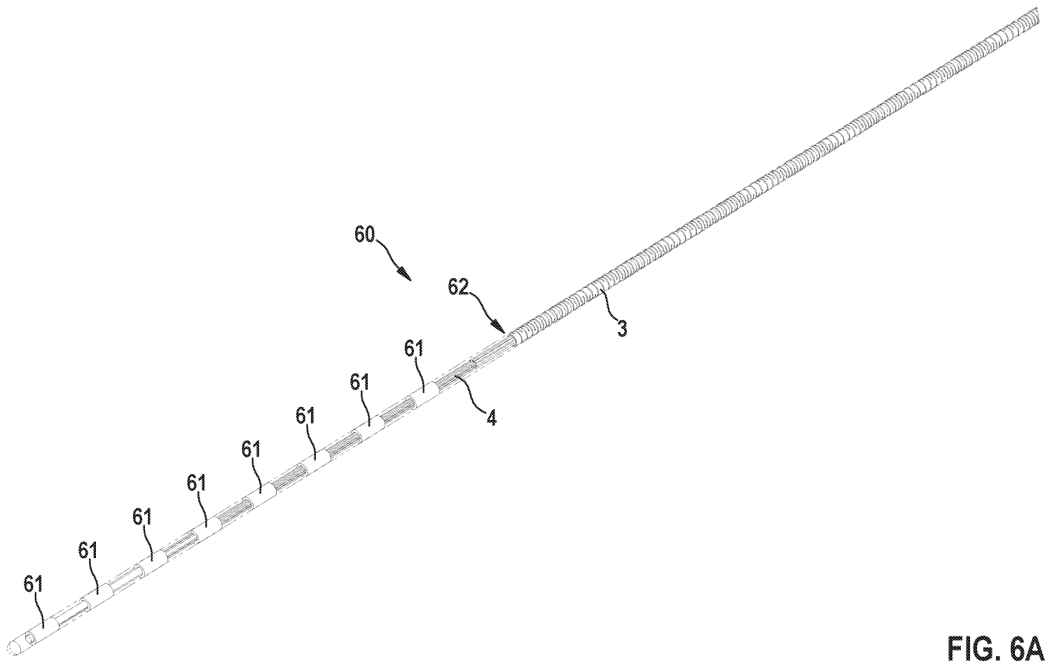

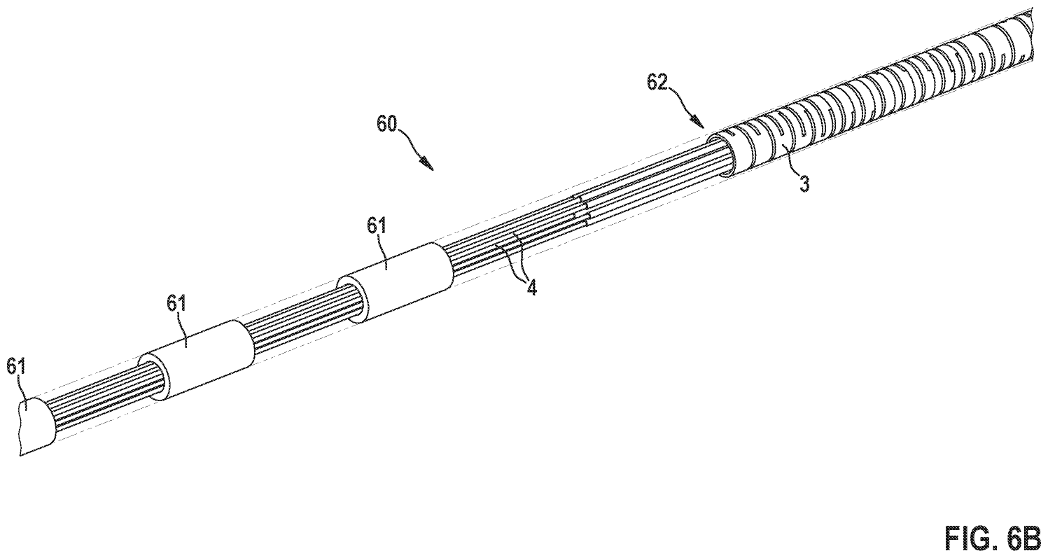

FIG. 6A shows the distal end of a lead electrode 60 with eight electrodes 61, each connected to a conductor 4. Note that in FIG. 6A and FIG. 6B, the conductors from the cross section of the lead electrode is shown, wherein other components like insulating material etc. as depicted in FIG. 2, are depicted translucently for illustrative purposes. Shield 3 is arranged around the lead electrode, wherein the distal end of the shield 62 is located such that none of the electrodes 61 is covered by shield 3. The shown embodiment is an exemplary implementation of the present invention, Technological variations within the considerations of the skilled person, as for instance the number of features, variations of dimensions, different materials, are incorporated in the scope of the present disclosure.

FIG. 6B shows a close up of lead electrode from FIG. 6a in the section between distal end of shield 3 and the electrodes 61 at the distal end of lead 60.

The present invention allows MRI shielding with varying flexibility along the length of the lead electrode.

Moreover, the proposed MRI shielding also provides mechanical shielding against pinch points, stresses at anchor locations, and protection from conductor externalization. The design is less complex and established a single component shielding solution. Furthermore, the design allows for integrated fluoro markers. Finally, the lead electrode according to the present invention is less bulky than other solutions.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

1. A lead electrode, comprising:
a lead body extending along a longitudinal axis (x) of the lead electrode;
a tubular shield arranged around at least a portion of said lead body, said shield being configured to protect said lead body from electromagnetic interference;
said shield being formed with a plurality of slots arranged along the longitudinal axis of the lead electrode; or
said shield being formed with at least one helical slot which extends along a helical path along the longitudinal axis.

2. The lead electrode according to claim 1, wherein respective said slots extend in a circumferential direction of said shield, or wherein respective said slots (30, 31) comprise at least one slot having a shape of a circle segment, or wherein respective said slots comprise a helical slot, or wherein the respective said slots comprise interrupted helical slots.

3. The lead electrode according to claim 1, wherein said slots are grouped in pairs of slots, said pairs are arranged side by side along the longitudinal axis, each pair of slots comprises a first and a second slot, and wherein said first and second slot are arranged side by side in the circumferential direction.

4. The lead electrode according to claim 1, wherein said tubular shield is formed with at least two slots each extending in the circumferential direction of said shield 3 and each forming an arc of less than 180 degrees.

5. The lead electrode according to claim 1, wherein said slots are laser-cut slots cut into said shield, or wherein said slots are etched into said shield.

6. The lead electrode according to claim 1, wherein said shield comprises or is formed from a material selected from the group consisting of a stainless steel, an alloy comprising Ni, Co, Cr, and Mo, cobalt-chromium, gold, tantalum, nitinol.

7. The lead electrode according to claim 1, wherein said shield comprises an outer surface that is coated with a material having low electrical resistance, and/or wherein said shield comprises an inner surface facing said lead body and coated with a material having low electrical resistance.

8. The lead electrode according to claim 7, wherein said material having low electrical resistance is gold, and/or wherein one or both of said outer surface and said inner surface of said shield is electroplated with said material having low electrical resistance.

9. The lead electrode according to claim 1, wherein said lead electrode comprises an electrically insulating layer arranged on said shield.

10. The lead electrode according to claim 9, wherein said electrically insulating layer is formed with at least one through-opening to expose a section of said shield and to allow dissipation of RF energy induced during magnetic resonance imaging of said lead electrode.

11. The lead electrode according to claim 9, wherein said electrically insulating layer comprises, or is formed of, one of the materials selected from the group consisting of polyurethane, silicone, silicon carbide, silicone-urethane copolymer, parylene, ethylene tetrafluoroethylene, and polytetrafluoroethylene.

12. The lead electrode according to claim 1, wherein said tubular shield comprises at least one marker for identifying the lead electrode, said marker being configured to be visible under fluoroscopy.

13. The lead electrode according to claim 1, wherein said lead body is formed out of, or comprises, an electrically insulating material.

14. The lead electrode according to claim 13, wherein said electrically insulating material is, or comprises, a material selected from the group consisting of polyurethane, silicone, and silicone-urethane copolymer.

15. The lead electrode according to claim 1, wherein said lead body is formed with a center lumen configured to receive a longitudinal element to enable said element to slide in said center lumen.

16. The lead electrode according to claim 1, wherein said lead body is formed with a plurality of lumens each extending along the longitudinal axis.

17. The lead electrode according to claim 1, further comprising a plurality of conductors.

18. The lead electrode according to claim 17, wherein each of said conductors is coated with an electrical insulation material selected from the group consisting of ethylene tetrafluoroethylene, perfluoroalkoxy alkanes, and polytetrafluoroethylene.

19. The lead electrode according to claim 18, further comprising a plurality of conductors each extending in a respective lumen formed in said lead body along the longitudinal axis.

20. The lead electrode according to claim 1, wherein said lead body and said shield have a length in the direction of the longitudinal axis that lies in the range from 35 cm to 95 cm.
